(19) United States
(12) Patent Application Publication
(10) Pub. No.: US 2006/0039898 A1
(43) Pub. Date: Feb. 23, 2006

(54) Method of skin care and/or treatment using rice (Oryza sativa) hemoglobin or other hexacoordinated, non-symbiotic plant hemoglobins

(76) Inventors: Hannah Naomi Sivak, Gilbert, AZ (US); Alberto Alvaro Iglesias, Rosario (AR); Miguel Angel Ballicora, Skokie, IL (US)

Correspondence Address: Hannah Naomi Sivak, 1213 W. Seascape Dr., Gilbert, AZ, 85233, US

(21) Appl. No.: 11/190,605

(22) Filed: Jul. 27, 2005

Related U.S. Application Data
(60) Provisional application No. 60/591,340, filed on Jul. 27, 2004.

(51) Int. Cl. A61K 38/41; A61K 38/44; A61K 38/05; A61K 31/375; A61K 31/355; A61K 31/015

(52) U.S. Cl. 424/94.4; 514/6; 514/18; 514/458; 514/474; 514/763

(57) ABSTRACT

Methods for the prevention and treatment of skin damage arising from exposure to nitric oxide and other pollutant oxidizing species, using rice (Oriza sativa) hemoglobin or other plant hexacoordinated, non-symbiotic hemoglobins, in a dermatologically acceptable carrier that can be topically applied to the skin areas, including lips. In some embodiments, other proteins such as superoxide dismutase and/or catalase or antioxidants are included.

### RELATED APPLICATION DATA

This application claims the benefit of U.S. Provisional Application No. 60/591,340, filed on Jul. 27, 2004.

### BACKGROUND OF THE INVENTION

Life in the city exposes skin to relatively high concentrations of pollutants, which originate mostly from car exhausts. Some states with large metropolis, like California and Arizona, are making efforts to reduce emissions, but even if these efforts become largely successful, air pollution in cities is a problem likely to will be with us for a long time.

The effect of air pollution on the respiratory system is well known, and an increase in the number of asthma sufferers in the last decades has been attributed, at least partly, to this problem. The effect of pollution on skin has less dramatic consequences than impaired breathing, but it is likely to have long term consequences.

Motor vehicles produce exhaust gases containing oxides of nitrogen such as nitric oxide (NO), because at the high temperatures in the cylinder, the car's combustion chamber, nitrogen and oxygen from the air react to form NO.

NO, because of its unpaired electron, is classified as a free radical and displays important reactivity with certain types of proteins and other free radicals. Free radicals trigger many reactions that produce hydroxyl, superoxide and other free radicals, which can damage DNA and other macromolecules. This kind of molecular damage contributes to aging, inflammatory disease, cancer, etc.

Reactive oxygen species are also produced endogenously. For example, hydrogen peroxide is part of the human immune response to a bacterial attack, and nitric oxide is used by the body as an internal signal. The same is true for plants, which in response to pathogens also produce an oxidative burst composed of superoxide, hydrogen peroxide and other reactive oxygen species whose production involves nitric oxide. Because of the very reactive nature of these signals and defensive mechanisms, the organism requires strict control of these endogenous reactive species, to prevent collateral damage from mechanisms that should only benefit the organism.

Myoglobin reacts with nitric oxide (Brunori, M, 2001 a, b), but it has been proposed that it is not myoglobin but the hexacoordinate hemoglobins that are in charge of preventing the collateral damage caused by endogenous reactive oxidative radicals. Indeed, there is extensive circumstantial evidence for this role (Kundo, S. et al., 2003. The discovery of hexacoordinate neuroglobin and histoglobin (also known as cytoglobin) in humans and other animals, suggest that hexacoordinate hemoglobins are common to a diverse group of living organisms and might even be ubiquitous.

Plant hexacoordinated hemoglobins should not be confused with leghemoglobin, a protein that controls the oxygen concentration reaching the nitrogen-fixing endosymbiotic bacteria present in the nodules, and is part of the group pf pentacoordinate hemoglobins, with their open binding site at the heme available for exogenous ligands.

### DESCRIPTION OF THE INVENTION

The objective of this invention is to provide a method and composition for a preventive regimen and/or therapy of skin based upon the topical application to exposed or affected skin areas of at least one active agent, in association with a dermatologically acceptable carrier or vehicle. This invention is based upon the finding that hemoglobins can react with NO and that plant hexacoordinated hemoglobins may be particularly suitable for the role of diasrming active oxidative species, including NO. Hexacoordinated hemoglobins may also increase the efficacy of other ingredients in topical compositions for the prevention and treatment of damage involving active oxidative species.

These and other objectives are accomplished by the present invention, which provides methods and compositions for the prevention and/or treatment of skin damage caused by active oxidative species like NO present in polluted environments or by any kind of stress leading to the formation of free radicals, by applying topically to the exposed or affected skin areas an effective amount of plant hexacoordinated hemoglobins, preferably in a dermatologically acceptable carrier.

As used herein, the term “plant hexacoordinated hemoglobins” encompasses hexacoordinated hemoglobins from any plant, fungi or blue-green algae purified from natural sources such as rice roots or obtained by genetic engineering means, i.e., by overexpression of a protein, natural or foreign to the organism used for its production.

Plant hexacoordinated hemoglobins obtained by overexpression in yeast or bacteria are preferred because they are less expensive than those obtained by purification from natural materials such as rice roots.

Many embodiments incorporate at least one other active ingredient with the plant hexacoordinated hemoglobins. These include natural or synthetic antioxidant molecules such as reduced glutathione, tocotrienols, vitamin E, ascorbic acid, astaxanthin, and/or lycopene. Other desirable ingredients are proteins capable of alleviating oxidative stress such as catalase and/or superoxide dismutase.

In the preferred practice of the invention, a plant hexacoordinated hemoglobin preparation is applied in admixture with a dermatologically acceptable carrier or vehicle (e.g., as a lotion, cream, ointment, serum) so as to facilitate topical application and, in some cases, provide additional therapeutic effects as might be brought about by moisturizing the affected skin areas. As noted, other ingredients are advantageously included in the compositions.

The amount of plant hexacoordinated hemoglobin necessary to bring about prevention and/or therapeutic treatment of damage by oxidative stress is not fixed, and is dependent upon the source, purity and activity of the protein employed, the amount and type of any additional ingredients used, particularly those that appear to exhibit synergistic effects, the skin type of the user, and, where present, the severity and extent of skin damage or the degree of pollution of the environment to which the skin will be exposed. Generally, the plant hexacoordinated hemoglobin or composition containing it is topically applied in effective amounts to skin areas which have been damaged, or which are susceptible to damage, because of exposure to pollutants.

In one embodiment, the composition contains from about 0.0001% to about 1% (weight per volume), preferably from more than 0.001% or 0.002% to about 0.01% plant hexacoordinated hemoglobin.

While the carrier for plant hexacoordinated hemoglobin can be very simple (such as saline solution), it is generally preferred that the carrier be a composition that will facilitate topical application, and particularly one which will form a film or layer on the skin to which it is applied so as to localize the active ingredient. Many such compositions are known in the art, and can take the form of lotions, creams, gels, etc. Typical compositions include lotions containing water and/or alcohols and emollients such as natural oils and waxes, silicone oils, hyaluronic acid, glyceride derivatives, fatty acids or fatty acid esters or alcohols or alcohol ethers, lanolin and derivatives, polyhydric alcohols or esters, wax esters, sterols, phospholipids and the like, and generally also emulsifiers (nonionic, cationic or anionic), although some of the emollients inherently possess emulsifying properties. These same general ingredients can be formulated into a cream rather than a lotion, or into gels, or into solid sticks by utilization of different proportions of the ingredients and/or by inclusion of thickening agents such as gums or other forms of hydrophilic colloids. Such compositions are referred to herein as dermatologically-acceptable carriers.

Many preferred embodiments of this invention contain at least one or two, and sometimes several, other active ingredients in addition to plant hexacoordinated hemoglobins, provided that the ingredients are not acids present in concentrations high enough to denature and inactivate plant hexacoordinated hemoglobins, which are proteins that can be denatured by exposure to extreme conditions of pH, temperature, etc.

Reduced glutathione, tocotrienol, lycopene, astaxanthin, ascorbic acid, and/or vitamin E may also be added to the plant hexacoordinated hemoglobins composition, alone or in combination with other ingredients in some embodiments.

In terms of a possible explanation for the effectiveness of the active ingredients in the prevention or treatment of damage to the skin, it is noted that plant hexacoordinated hemoglobins will help scavenge free radicals such as the NO present in polluted environments. Just like in the living cell, where a number of antioxidants work in a concerted fashion, some embodiments of this invention also use the synergistic effect of antioxidants.

The method of the present invention is particularly useful for the prevention and treatment of damage by reactive oxygen species. Plant hexacoordinated hemoglobins, alone or with other active ingredients can thus be added to dermatological creams and emollients as well as to commercial sunscreens to enhance their protective activity.

Having described the invention with reference to particular compositions, theories of effectiveness, it will be apparent to those of skill in the art that it is not intended that the invention be limited by such illustrative embodiments or mechanicisms, and that modifications can be made without departing from the scope or spirit of the invention, as defined by the appended claims. It is intended that all modifications and variations be included within the scope of the invention. The claims are meant to cover the claimed components and steps in any sequence which is effective to meet the objectives there intended, unless the context specifically indicates the contrary.

### RELEVENT REFERENCES

Please note that we found no references to the use of hexacoordinated plant hemoglobins in dermatological compositions.
Brunori, M. Nitric oxide, cytochrome-c oxidase, and myoglobin.
Trends Biochem. Sci. (2001a) 26, 21-23.
Brunori, M. Nitric oxide moves myoglobin centre stage. Trends Biochem. Sci. (2001b) 26, 209-210.
Doherty, D. et al. The rate of reaction with NO determines the hypertensive effect of cell-free hemoglobin. Nat. Biotechnol. (1998) 16, 672-676.
Flogel, U. et al. Myoglobin: a scavenger of bioactive NO. Proc. Natl. Acad. Sci. U.S.A. (2001) 98, 735-740.
Kundu, S. et al. Plants, humans and hemoglobins. Trends in Plant Science (2003) 8, 391-

[symbol]

We claim:

1. A method for the prevention and treatment of skin damage induced by exposure to pollutants or to stressors leading to the formation of active oxygen species which consists of applying a composition containing plant hexacoordinated hemoglobins in a dermatologically acceptable carrier to the affected skin area.

2. A method for the treatment of skin damage induced by exposure to solar or other type of radiation or to stressors leading to the formation of active oxygen species which consists of applying a composition containing plant hexacoordinated hemoglobins to the affected skin area.

3. A method in accordance with claims 1 or 2, wherein said composition further comprises one or more additional ingredients selected from the group consisting of tocotrienols, vitamin E, ascorbic acid, superoxide dismutase, catalase, astaxanthin, lycopene, reduced glutathione.
